(19) United States
(12) Patent Application Publication
(10) Pub. No.: US 2011/0129762 A1
(43) Pub. Date: Jun. 2, 2011

(54) METHOD OF INCREASING HYDROPHILIC PROPERTY OF CRYSTALLINE CARBON USING SURFACE MODIFIER AND METHOD OF PREPARING PLATINUM CATALYST USING THE SAME

(75) Inventors: Ki Sub Lee, Seoul (KR); Bumwook Roh, Yongin (KR); Han Sung Kim, Seoul (KR); Hyung-Suk Oh, Incheon (KR)

(73) Assignees: HYUNDAI MOTOR COMPANY, Seoul (KR); KIA MOTORS CORPORATION, Seoul (KR); Industry-Academic Cooperation Foundation, Younsei University, Seoul (KR)

(21) Appl. No.: 12/751,411

(22) Filed: Mar. 31, 2010

(30) Foreign Application Priority Data
Nov. 30, 2009 (KR) 10-2009-0117213

(51) Int. Cl. H01M 4/583; B01J 21/18; H01M 4/90; C07C 63/44; C07C 63/36; C07C 229/56; C07D 211/06; C07C 321/26; C07D 235/28

(52) U.S. Cl. 429/524; 502/185; 252/182.1; 562/405; 562/490; 562/457; 546/197; 568/67; 548/307.1; 977/742; 977/734; 977/762

(57) ABSTRACT

The present invention features a method for increasing hydrophilic properties of crystalline carbon using a surface modifier and a method for preparing a Pt/C catalyst using the same. In certain preferred embodiments, the present invention features a method for increasing hydrophilic properties of crystalline carbon having water repellency by forming π-π interaction between the surface of the crystalline carbon and a surface modifier and a method for preparing a catalyst by supporting platinum (Pt) on the crystalline carbon having increased hydrophilic property. The Pt/C catalyst prepared by the methods of the present invention is useful for the preparation of electrode materials for fuel cells.

### CROSS-REFERENCE(S) TO RELATED APPLICATIONS

This application claims under 35 U.S.C. §119(a) the benefit of Korean Patent Application No. 10-2009-0117213, filed on Nov. 30, 2009, the entire contents of which are incorporated herein by reference.

### BACKGROUND OF THE INVENTION

1. Field of the Invention

The present invention relates, generally, to a method for increasing hydrophilic property of crystalline carbon using a surface modifier and a method for preparing a platinum (Pt)/C catalyst using the same. Preferably, the crystalline carbon having increased hydrophilic property enables easier supporting of platinum (Pt) and is preferably used in the preparation of fuel cell electrode materials.

2. Description of Related Art

A fuel cell, which transforms the chemical energy resulting from the oxidation of the fuel directly into the electrical energy, has been called the next-generation energy source. Particularly, in the automobile-related fields, research has been directed to fuel cells because of their advantages in improved fuel efficiency, reduced emission, environmental friendliness, etc. In particular, research has focused on catalysts for oxidation and reduction reactions occurring in fuel cell electrodes.

In particular, research has been focused on the preparation of platinum (Pt) into nanoparticles or to support well dispersed Pt on carbon having high specific surface with high content in order to suitably improve catalytic activity of fuel cells (J. Power Sources, 139, 73). At present, carbon black is generally used as a support for Pt, but its durability deteriorates because of corrosion of carbon in the course of operation of the fuel cell (J. Power Sources, 183, 619). Accordingly, research on fuel cell catalysts using crystalline carbon with superior electrical and physical properties, such as carbon nanotube (CNT), carbon nanofiber (CNF) as a support have been actively carried out (J. Power Sources, 158, 154).

However, CNT and CNF are difficult to apply for preparation of a high-content, highly dispersed Pt/C catalyst since they tend to agglomerate in a polar solvent because of surface water repellency (Electrochim. Acta. 50, 791). Accordingly, functional groups are attached after oxidizing the surface of carbon support by means of plasma, air or strong acid. However, since plasma and air may result in a strong oxidation enough to destroy the surface structure of CNT or CNF, their application to a catalyst support may lead to deterioration of durability (Adv. Mater., 7, 275). Further, the acid treatment using strong acid such as nitric acid or sulfuric acid (Chem. Eur. J., 8, 1151) may also lead to destruction of the crystalline carbon structure and deterioration of durability during the acid treatment procedure. Thus, its application to a fuel cell catalyst support may result in increased electrochemical corrosion.

A number of methods to increase hydrophilic property without destruction of the surface structure of crystalline carbon have been proposed (J. Mater. Chem., 18, 1977, J. Am. Chem. Soc. 123, 3838, Korean Patent Application Publication No. 2006-084785 [symbol] [symbol] [symbol] US Patent Application Publication No. 2004-115232 [symbol] [symbol] [symbol] Korean Patent Application Publication No. 2009-079935 and US Patent Application Publication No. 2007-298168, all of which are incorporated by reference in their entireties herein). These methods aim at increasing hydrophilic property of the CNT surface using pyrene compounds. In particular, an attempt to uniformly arrange Pt, cadmium sulfide (CdS) and silica particles on CNT using 1-aminopyrene is disclosed in Adv. Funct. Mater. 16, 2431. Although these methods are effective in improving hydrophilic property of CNT, they are not suitably applicable to preparation of Pt/C catalysts because water dispersibility decreases with time.

The above information disclosed in this the Background section is only for enhancement of understanding of the background of the invention and therefore it may contain information that does not form the prior art that is already known in this country to a person of ordinary skill in the art.

### SUMMARY OF THE INVENTION

The present invention features a high-content, highly dispersed Pt/C catalyst that is preferably prepared by suitably increasing hydrophilic property of crystalline carbon by forming noncovalent π-π interaction between a surface modifier and the crystalline carbon and using the crystalline carbon as a support of a fuel cell catalyst.

Accordingly, present invention preferably provides a method for increasing hydrophilic property of crystalline carbon using a surface modifier.

The present invention further provides a method for preparing a catalyst by supporting platinum (Pt) on the crystalline carbon with increased hydrophilic property.

In preferred embodiments, the present invention preferably provides a method for suitably increasing hydrophilic property of crystalline carbon using a surface modifier.

In other preferred embodiments, the present invention also preferably provides a method for preparing a Pt/C catalyst, including: increasing hydrophilic property of crystalline carbon using a surface modifier; supporting Pt on the crystalline carbon to suitably prepare a catalyst; and washing and drying thus prepared catalyst to remove unwanted organic materials.

Preferably, the method for increasing hydrophilic property of crystalline carbon using a surface modifier and the method for preparing a Pt/C catalyst using the same according to the present invention enable preparation of a high-content, highly dispersed, highly durable Pt nanocatalyst wherein crystalline carbon has suitably increased hydrophilic property by π-π interaction between the crystalline carbon and the surface modifier and is resistant to electrochemical corrosion. Preferably, the catalyst may be usefully used, for example, as electrode material of fuel cells.

It is understood that the term “vehicle” or “vehicular” or other similar term as used herein is inclusive of motor vehicles in general such as passenger automobiles including sports utility vehicles (SUV), buses, trucks, various commercial vehicles, watercraft including a variety of boats and ships, aircraft, and the like, and includes hybrid vehicles, electric vehicles, plug-in hybrid electric vehicles, hydrogen-powered vehicles and other alternative fuel vehicles (e.g. fuels derived from resources other than petroleum).

As referred to herein, a hybrid vehicle is a vehicle that has two or more sources of power, for example both gasoline-powered and electric-powered.

The above features and advantages of the present invention will be apparent from or are set forth in more detail in the accompanying drawings, which are incorporated in and form a part of this specification, and the following Detailed Description, which together serve to explain by way of example the principles of the present invention.

### BRIEF DESCRIPTION OF THE DRAWINGS

The above and other objects, features and advantages of the present invention will become apparent from the following description of preferred embodiments given in conjunction with the accompanying drawings, in which:

FIG. 1 schematically illustrates formation of π-π interaction between 1-pyrenecarboxylic acid (1-PCA) and the surface of crystalline carbon;

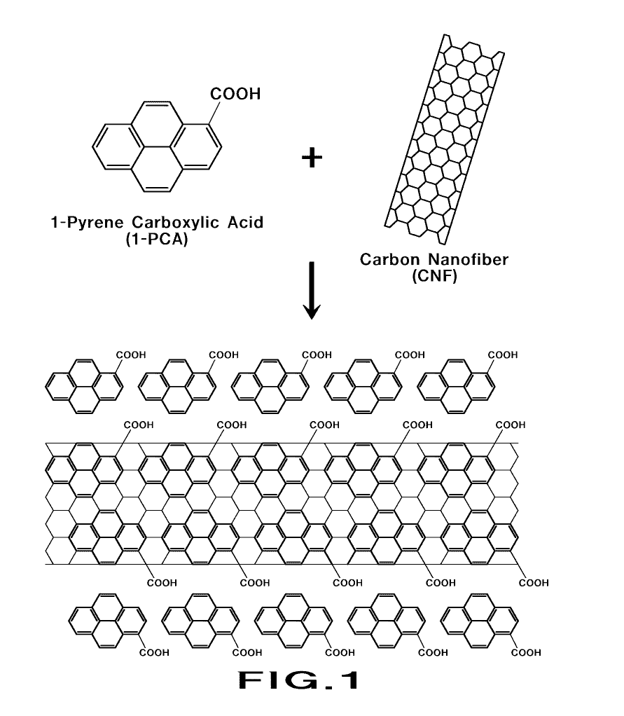

FIG. 2 shows a result of water dispersibility test for measuring hydrophilic property of carbon nanofiber (CNF) (a) and 1-PCA treated CNF (b);

FIG. 3 shows a result of water dispersibility test for measuring hydrophilic property of 1-PCA treated carbon nanocage (CNC) (a) and 1-aminopyrene (1-AP) treated CNC (b);

FIG. 4 shows high resolution transmission electron microscopy (HR-TEM) images of catalysts prepared by supporting platinum (Pt) on a herringbone CNF support treated or untreated with 1-PCA (magnification: 200,000×);

FIG. 5 shows HR-TEM images of catalysts prepared by supporting Pt on a platelet CNF support treated or untreated with 1-PCA (50,000×);

FIG. 6 shows HR-TEM images of Pt/CNC (a), 1-AP treated Pt/CNC (b) and 1-PCA treated Pt/CNC (c) (200,000×);

FIG. 7 shows X-ray diffraction patterns of catalysts prepared by supporting Pt on a herringbone CNF support treated or untreated with 1-PCA;

FIG. 8 shows performance of unit cells of catalysts prepared by supporting Pt on a herringbone CNF support treated or untreated with 1-PCA under air condition;

FIG. 9 shows performance of membrane electrode assemblies (MEAs) of catalysts prepared by supporting Pt on an untreated herringbone CNF support (a) or on a 1-PCA treated herringbone CNF support (b) under oxygen condition, before and after corrosion; and

FIG. 10 shows generation of CO₂ resulting from corrosion of catalysts prepared by supporting Pt on a herringbone CNF support treated or untreated with 1-PCA.

It should be understood that the appended drawings are not necessarily to scale, presenting a somewhat simplified representation of various preferred features illustrative of the basic principles of the invention. The specific design features of the present invention as disclosed herein, including, for example, specific dimensions, orientations, locations, and shapes will be determined in part by the particular intended application and use environment.

### DESCRIPTION OF SPECIFIC EMBODIMENTS

As described herein, the present invention includes a method for increasing the hydrophilic properties of crystalline carbon using a surface modifier.

In one embodiment, π-π interaction is formed between the surface modifier and the crystalline carbon and hydrophilic properties are provided by a hydrophilic functional group of the surface modifier.

In another embodiment, the surface modifier is one selected from 1-pyrenecarboxylic acid, 9-anthracenecarboxylic acid, fluorene-1-carboxylic acid, 1-pyrenebutyric acid, naphthoic acid, 1-pyreneacetic acid, naphtho-2-aminopyridine-3-carboxylic acid, 1,4-benzodioxane-6-carboxylic acid, 2-mercaptobenzimidazole, 2-naphthalenethiol, 1-mercaptopyrene, 6-mercaptobenzopyrene and 1,4-benzenedithiol.

In another further embodiment, the crystalline carbon is one selected from carbon nanotube, carbon nanofiber, carbon nanocoil and carbon nanocage.

In another aspect, the present invention features a method for preparing a Pt/C catalyst, comprising increasing hydrophilic property of crystalline carbon using a surface modifier; supporting platinum (Pt) on the crystalline carbon to prepare a catalyst; and washing and drying thus prepared catalyst to remove unwanted organic materials.

In another embodiment, the present invention features a Pt/C catalyst prepared by a method described in any one of the above-mentioned aspects herein.

In another further embodiment, the present invention features a fuel cell electrode comprising the catalyst according to a method described in any one of the above-mentioned aspects herein.

In still another embodiment, the present invention features a fuel cell comprising the electrode according to a method described in any one of the above-mentioned aspects herein.

Certain advantages, features and aspects of the present invention will become apparent from the following description of the embodiments with reference to the accompanying drawings, which is set forth hereinafter.

In certain preferred embodiments, the present invention provides a method for increasing hydrophilic property of crystalline carbon using a surface modifier.

In preferred embodiments, the crystalline carbon may include carbon nanotube (CNT), carbon nanofiber (CNF), carbon nanocoil, carbon nanocage (CNC), etc. Preferably, the aforementioned tend to agglomerate in a polar solvent because of water repellency. Preferably, to address the issue of water repellency, hydrophilic property of the surface of the crystalline carbon may be suitably increased by forming π-π interaction between the aromatic surface of the crystalline carbon and a surface modifier having an hydrophilic functional group such as carboxyl (—COOH) or thiol (—SH). In certain preferred embodiments, the surface modifier may be an aromatic cyclic compound having a carboxyl or thiol group, such as, but not limited to, 1-pyrenecarboxylic acid (1-PCA), 9-anthracenecarboxylic acid, fluorene-1-carboxylic acid, 1-pyrenebutyric acid, naphthoic acid, 1-pyreneacetic acid, naphtho-2-aminopyridine-3-carboxylic acid, 1,4-benzodioxane-6-carboxylic acid, 2-mercaptobenzimidazole, 2-naphthalenethiol, 1-mercaptopyrene, 6-mercaptobenzopyrene and 1,4-benzenedithiol. Preferably, one selected from 1-PCA, 9-anthracenecarboxylic acid, fluorene-1-carboxylic acid, 1-pyrenebutyric acid, naphthoic acid, 2-mercaptobenzimidazole and 2-naphthalenethiol is used. More preferably, 1-PCA, which is represented by Chemical Formula 1, shown below, is used.

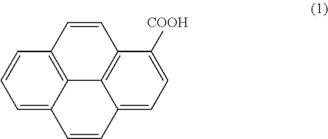

In exemplary preferred embodiments, the crystalline carbon and the surface modifier are stirred in an ethanol solvent. Preferably, the crystalline carbon is recovered using a filtering apparatus and suitably dried to obtain crystalline carbon with increased hydrophilic property.

In certain preferred embodiments, the present invention also provides a method for preparing a Pt/C catalyst, comprising: increasing hydrophilic property of crystalline carbon by suitably treating the surface of the crystalline carbon with a surface modifier; supporting platinum (Pt) on the crystalline carbon; and washing and drying thus prepared catalyst.

Preferably, the procedure of increasing hydrophilic property of the crystalline carbon using the surface modifier is the same as described above.

According to further preferred embodiments of the present invention, polyol process is employed to suitably prepare the catalyst by supporting Pt on the crystalline carbon. Preferably, in the polyol process, ethylene glycol is used at once as a solvent and as a reducing agent. Preferably, glycolate anion produced as ethylene glycol is oxidized, acts as a stabilizer, and maintains Pt particles at nano size. In further preferred embodiments, sodium hydroxide is added to ethylene glycol to keep pH at 12 or higher. In further preferred embodiments, an adequate amount of Pt precursor is added to a solvent and stirred. Preferably, the Pt precursor may be platinum chloride, potassium tetrachloroplatinate, tetraammineplatinum chloride, etc. In further preferred embodiments, after the crystalline carbon treated with the surface modifier is added to the solvent, the temperature is raised to 160° C. while sufficiently stirring. Preferably, during this procedure, the Pt precursor is suitably reduced as ethylene glycol is oxidized. Preferably, the glycolate anion produced as ethylene glycol is oxidized and prevents agglomeration of the reduced Pt particles. In further preferred embodiments, after the reaction is suitably completed, the temperature is decreased to room temperature and the solution is sufficiently stirred.

Preferably, thus prepared catalyst is suitably washed and dried to remove unwanted organic materials. Preferably, during this procedure, organic acids and other impurities produced during oxidation of ethylene glycol are suitably removed by sufficiently washing with ultrapure water and drying. As a result, the Pt/C catalyst is obtained as powder.

In accordance with preferred methods for preparing a Pt/C catalyst of the present invention as described herein, hydrophilic property of the crystalline carbon is suitably increased by forming π-π interaction between the surface of the crystalline carbon and the surface modifier. Accordingly, a high-content, highly dispersed, highly durable Pt nanocatalyst resistant to electrochemical corrosion may be suitably prepared. Preferably, when used for an electrode of a fuel cell, the amount of the catalyst suitably coated on the electrode may be decreased. Accordingly, the electrode becomes thinner and has improved fuel transfer efficiency.

### EXAMPLES

The examples and experiments according to certain preferred embodiments of the present invention are now be described. The following examples are for illustrative purposes only and not intended to limit the scope of the present invention.

### Example 1

In a first example, 1-Pyrenecarboxylic acid (1-PCA, 100 mg) was added to ethanol (400 mL) and stirred for 30 minutes. Then, herringbone carbon nanofiber (CNF, 200 mg) was added to the 1-PCA solution and stirred for 6 hours. This is to form π-π interaction between the pyrene of 1-PCA and the graphene of CNF. 1-PCA treated CNF was suitably recovered by filtration under reduced pressure and dried in an oven at 40° C. for 30 minutes. 1-PCA treated CNF (144 mg) was added to ethylene glycol (25 mL) and stirred for 20 minutes. Then, to prepare a 40 wt % Pt/CNF catalyst, after adding 0.1 M sodium hydroxide (NaOH) solution (100 mL) and a Pt precursor PtCl₄ (150 mg), the mixture was stirred for 30 minutes. After carrying out a reaction at 160° C. for 3 hours under reflux to reduce the Pt precursor, the mixture was suitably cooled to room temperature and adjusted to pH 3 using sulfuric acid (H₂SO₄). Then, after exposing to air, the mixture was stirred for 12 hours. The reaction solution was filtered under reduced pressure to recover the prepared catalyst, which was washed several times with ultrapure water and dried in an oven at 160° C. for 30 minutes. As a result, the 1-PCA treated Pt/CNF catalyst was obtained.

### Example 2

In a second example, a catalyst was prepared in the same manner as Example 1, except for using platelet CNF instead of herringbone CNF.

### Example 3

In a third example, a catalyst was prepared in the same manner as Example 1, except for using carbon nanocage (CNC) instead of CNF.

### Comparative Example 1

Untreated Pt/CNF was prepared in the same manner as Example 1 by a polyol process without surface modification of CNF.

### Comparative Example 2

Untreated Pt/CNF was prepared in the same manner as Example 2 by a polyol process without surface modification of CNF.

### Comparative Example 3

Untreated Pt/CNC was prepared in the same manner as Example 3 by a polyol process without surface modification of CNC.

### Comparative Example 4

A catalyst was prepared in the same manner as Example 3, using 1-aminopyrene (1-AP) for surface modification of CNC.

### Test Example 1

### Testing of Increased Hydrophilic Property of 1-PCA Treated Crystalline Carbon

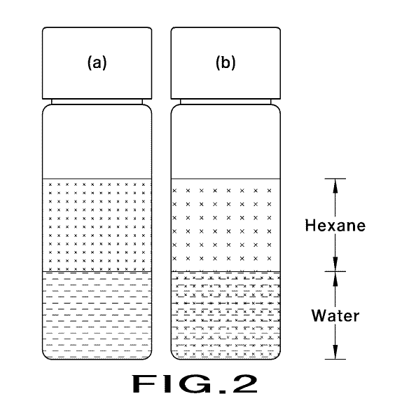

In order to test increased hydrophilic property of 1-PCA treated CNF prepared in Example 1, water dispersibility test was carried out. Untreated CNF was also tested for comparison. The results are shown in FIG. 2. After mixing CNF with water and then adding hexane, it was observed whether CNF was dispersed in the aqueous layer. As shown in FIG. 2, 1-PCA treated CNF (b) was dispersed well in water, differently from untreated CNF (a). This demonstrates that 1-PCA increases hydrophilic property of CNF.

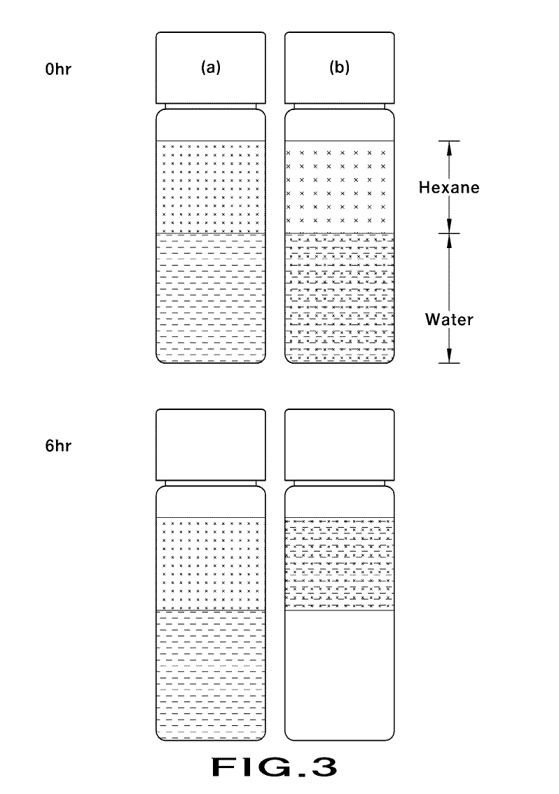

Also, water dispersibility test was carried out to test hydrophilic property of 1-PCA treated CNC (a) prepared in Example 3 and 1-AP treated CNC (b) prepared in Comparative Example 4. The results are shown in FIG. 3. When CNC with high water repellency was treated with 1-PCA or 1-AP, CNC was dispersed well in the aqueous layer because of increased hydrophilic property. However, 6 hours later, 1-AP treated CNC (b) moved to the hexane layer whereas 1-PCA treated CNC (a) remained in the aqueous layer. This result shows that 1-PCA treated CNC has higher hydrophilic property than 1-AP treated CNC.

### Test Example 2

### Testing of Particle Size and Dispersion of Supported Pt

Dispersion of Pt on the Pt/C catalysts prepared in Examples 1 to 3 and Comparative Examples 1 to 4 was tested by high resolution transmission electron microscopy (HR-TEM).

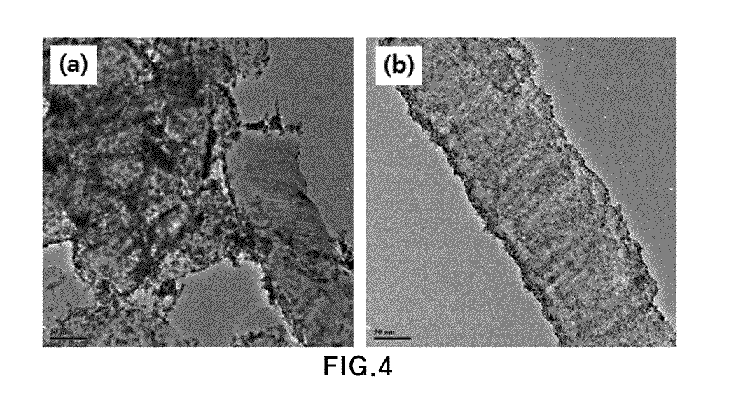

FIG. 4 (a) is an HR-TEM image of the catalyst prepared in Comparative Example 1. Pt particle size was 2.5 nm. FIG. 4 (b) is an HR-TEM image of the catalyst prepared in Example 1. Pt particle size was 2.5 nm. There was no difference in particle size. However, the catalyst treated with 1-PCA (FIG. 4 (b)) showed higher Pt density and better dispersion than FIG. 4 (a). It is because the carboxyl (—COOH) group of 1-PCA not only increases hydrophilic property of carbon but also acts as a conglutination site of Pt thereby leading to uniform supporting of Pt and increased supporting density thereof.

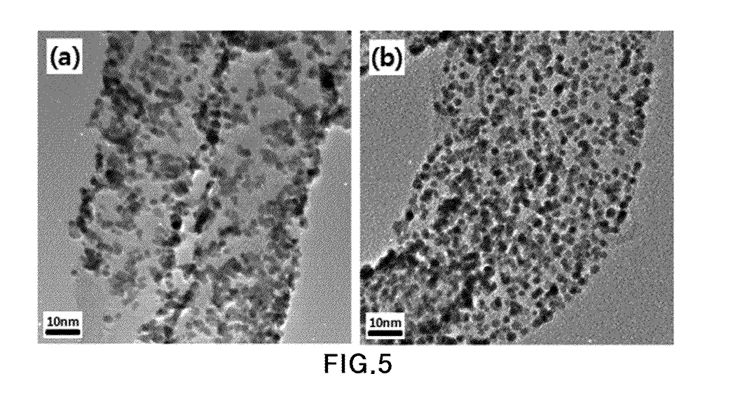

FIG. 5 (a) shows an HR-TEM image of the catalyst prepared in Comparative Example 2 and FIG. 5 (b) shows an HR-TEM image of the catalyst prepared in Example 2 (50,000×). In FIG. 5 (a), regions where Pt is not supported or Pt particles are agglomerated are observed. The 1-PCA treated catalyst (FIG. 5(b)) showed higher Pt density and better dispersion than FIG. 5(a). It is because the 1-PCA CNF has increased hydrophilic property while supporting of Pt on the 1-PCA untreated CNF is difficult because of water repellency.

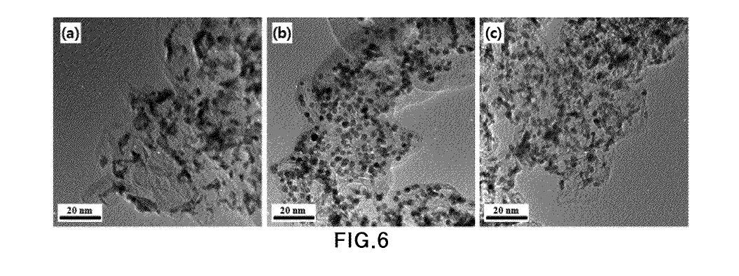

FIG. 6 shows HR-TEM images of the catalysts prepared in Comparative Example 3 (a), Comparative Example 4 (b) and Example 1 (c). The catalyst with water repellency (a) shows regions where Pt is not supported or Pt particles are agglomerated. The 1-AP treated catalyst (b) shows more uniform supporting of Pt than (a) but with increased Pt particle size. The 1-PCA treated catalyst (c) shows improved dispersion as (b) as well as small Pt particle size as (a).

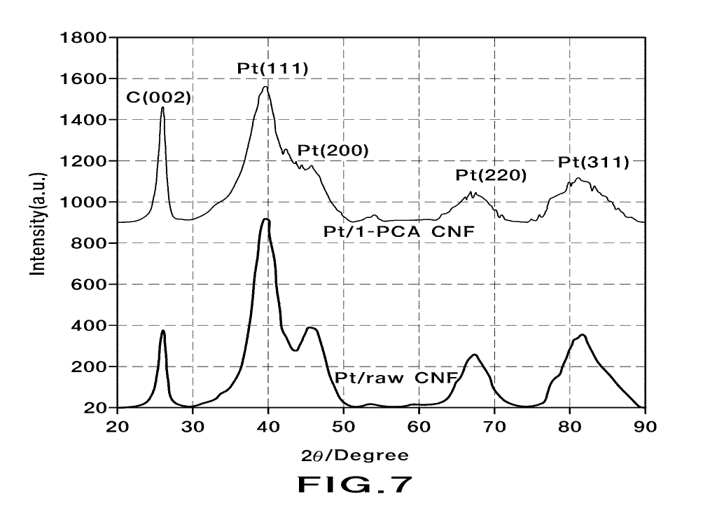

FIG. 7 shows X-ray diffraction patterns of the catalysts prepared in Comparative Example 1 and Example 1. Pt particle size was calculated using the Scherrer formula from the Pt (220) peak at 2θ=67°. Pt particle size was 2.0 nm for Comparative Example 1 and 1.8 nm for Example 1. Accordingly, it can be seen that Pt particle size was 0.2 nm smaller when 1-PCA treated CNF was used as the support.

Further, Pt particle size of the catalysts prepared in Comparative Examples 3 and 4 and Example 3 was calculated as 2.5 nm, 2.7 nm and 2.5 nm, respectively.

Accordingly, when compared with treatment with 1-AP, the treatment with 1-PCA enables easier improvement hydrophilic property of the water-repellent support and preparation of a high-content, highly dispersed Pt/C catalyst.

### Test Example 3

### Measurement of Pt Supporting Ratio and Catalytic Active Area

Inductively coupled plasma (ICP) and cyclic voltammetry (CV) experiments were performed to measure Pt supporting ratio and catalytic active area of the catalysts. Pt supporting ratio measured by ICP analysis was 23.9 wt % for Comparative Example 1 and 35.5 wt % for Example 1, 60% and 89% of the target value 40 wt %, respectively. Effective catalytic active area of the Pt catalyst measured by CV experiment was 50.3 m²/g Pt and 51.2 m²/g Pt, respectively. The catalytic active area was similar without regard to the 1-PCA treatment. This is because, as seen from the X-ray diffraction patterns and the HR-TEM images, the particle size of Pt supported on CNF differs only by 0.2 nm.

Comparative Examples 3 and 4 and Example 3 showed Pt supporting ratio of 35.0 wt %, 36.0 wt % and 40 wt %, respectively.

### Test Example 4

### Measurement of Performance of Unit Cell Under Air Condition

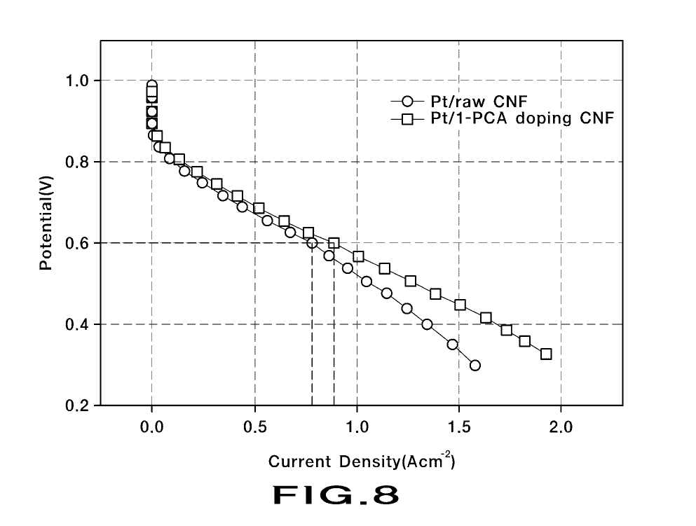

A fuel cell electrode was prepared using the catalyst prepared in Example 1 or Comparative Example 1 and performance of the unit cell was measured under air condition. The result is shown in FIG. 8. 1.5 stoic hydrogen was supplied to the anode, and 2 stoic air was supplied to the cathode. 1-PCA treatment (Example 1) resulted in better performance of 0.89 A/cm² at 0.6 V than 0.78 A/cm² of Comparative Example 1 under air condition, whereas similar performance was observed without regard to 1-PCA treatment under oxygen condition. This is because Pt supporting ratio of Example 1 (35.5 wt %), wherein the catalyst was treated with 1-PCA without growth of Pt particles, is higher than that of Comparative Example 1 (23.9 wt %), and, thus, the amount of the catalyst coated on the electrode decreases. Decreased coating amount of the catalyst on the electrode results in smaller electrode thickness and improved fuel transfer. As a result, the performance under air condition is improved.

### Test Example 5

### Catalyst Corrosion Test

Corrosion test was performed on the catalysts prepared in Example 1 and Comparative Example 1. Commercially available Johnson Matthey 40 wt % Pt/C catalyst was mixed with 5 wt % Nafion solution and coated on the anode side of N212 Nafion membrane at Pt 0.4 mg/cm². On the cathode side of the N212 Nafion membrane, the catalyst prepared in Comparative Example 1 was mixed with 5 wt % Nafion solution and coated at Pt 0.4 mg/cm². Then, after connecting a gas diffusion layer (GDL) and a gasket to the unit cell, corrosion test was carried out. Before starting corrosion, measurement of performance of the membrane-electrode assembly (MEA) under oxygen condition, impedance and CV was carried out. Then, a constant voltage of 1.4 V_{SHE} was supplied to the cathode for 30 minutes using a potentiostat to corrode the catalyst layer. The counter electrode and the reference electrode of the potentiostat were connected to the anode of the unit cell and the working electrode was connected to the cathode. Hydrogen was supplied to the anode at 20 mL/min and nitrogen was supplied to the cathode at 30 mL/min. The unit cell was maintained at 90° C. CO₂ resulting from the corrosion of the catalyst layer was measured in real time using a mass spectrometer. Upon completion of the corrosion, performance of the MEA, impedance and CV were measured to compare them with those before the corrosion. Based on the results, corrosion resistance of the catalyst was evaluated.

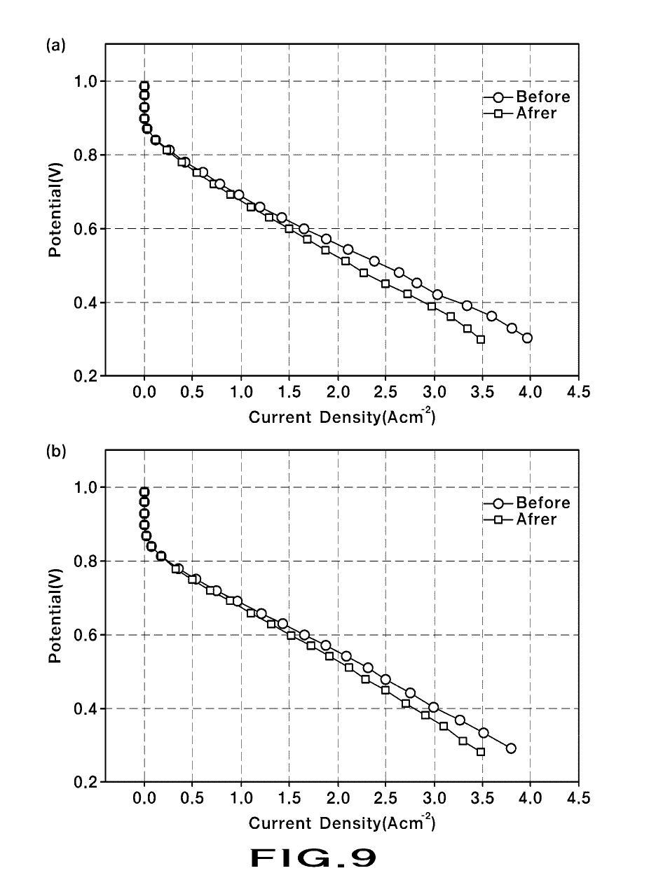

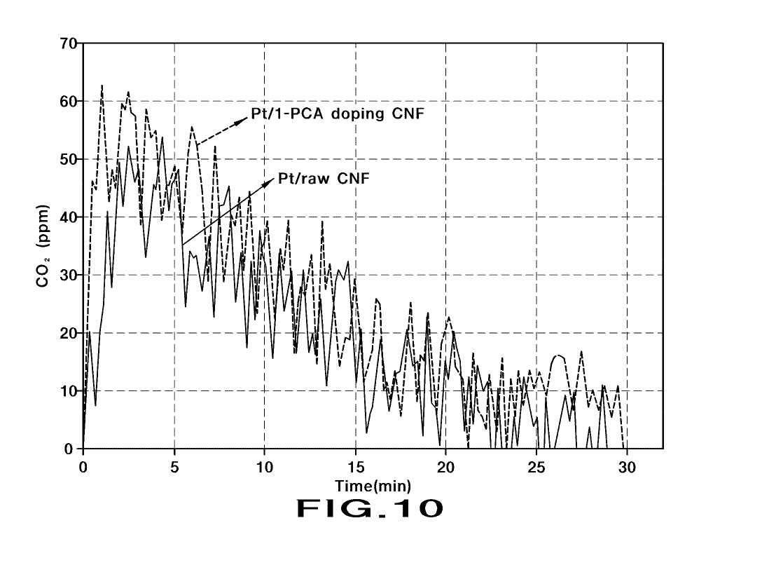

FIG. 9 and FIG. 10 show the corrosion test result of the catalysts prepared in Example 1 and Comparative Example 1. The result is summarized in Table 1, shown below.

TABLE 1

| | Performance of | | Active surface area | | | | |
|---|---|---|---|---|---|---|---|
| | MEA (A/cm²) | | (m²/g) | | Impedance (O cm²) | | CO₂ |
| | Before | After | Before | After | Before | After | production |
| | corrosion | corrosion | corrosion | corrosion | corrosion | corrosion | (μL) |
| Comp. | 1.66 | 1.50 | 32.5 | 31.7 | 0.0429 | 0.0458 | 18 μL |
| Ex. 1 | −9.6% | | −2.5% | | +6.8% | | |
| Ex. 1 | 1.67 | 1.51 | 30.1 | 28.8 | 0.0394 | 0.0403 | 19 μL |

| | | |
|---|---|---|
| −9.6% | −4.3% | +2.3% |

Unit cell performance under oxygen condition at 0.6 V before corrosion was 1.66 A/cm² for Comparative Example 1 and 1.67 A/cm² for Example 1. After corrosion of the cathode catalyst by supplying a voltage of 1.4 V_{SHE} to the cathode, unit cell performance at 0.6 V decreased by 9.6% for Comparative Example 1 (FIG. 9 (a)) and also by 9.6% for Example 1 (FIG. 9(b)). Active surface area decreased by 2.5% for Comparative Example 1 and by 4.3% for Example 1. Impedance increased by 6.8% for Comparative Example 1 and by 2.3% for Example 1. FIG. 10 shows a result of measuring generation of CO₂ resulting from corrosion of the Pt/C catalysts using a mass spectrometer. As shown in FIG. 10, there was no big difference between Comparative Example 1 (18 μL) and Example 1 (19 μL). These results show that the formation of π-π interaction on crystalline carbon using 1-PCA followed by the supporting of Pt has no significant effect on electrochemical corrosion.

While the present invention has been described with respect to the specific embodiments, it will be apparent to those skilled in the art that various changes and modifications may be made without departing from the spirit and scope of the invention as defined in the following claims.

What is claimed is:

1. A method for increasing the hydrophilic properties of crystalline carbon using a surface modifier.

2. The method for increasing hydrophilic properties of crystalline carbon according to claim 1, wherein π-π interaction is formed between the surface modifier and the crystalline carbon and hydrophilic property is provided by a hydrophilic functional group of the surface modifier.

3. The method for increasing hydrophilic property of crystalline carbon according to claim 1, wherein the surface modifier is one selected from the group consisting of: 1-pyrenecarboxylic acid, 9-anthracenecarboxylic acid, fluorene-1-carboxylic acid, 1-pyrenebutyric acid, naphthoic acid, 1-pyreneacetic acid, naphtho-2-aminopyridine-3-carboxylic acid, 1,4-benzodioxane-6-carboxylic acid, 2-mercaptobenzimidazole, 2-naphthalenethiol, 1-mercaptopyrene, 6-mercaptobenzopyrene and 1,4-benzenedithiol.

4. The method for increasing hydrophilic property of crystalline carbon according to claim 1, wherein the crystalline carbon is one selected from the group consisting of: carbon nanotube, carbon nanofiber, carbon nanocoil and carbon nanocage.

5. A method for preparing a Pt/C catalyst, comprising:
increasing hydrophilic property of crystalline carbon using a surface modifier;
supporting platinum (Pt) on the crystalline carbon to prepare a catalyst; and
washing and drying thus prepared catalyst to remove unwanted organic materials.

6. A Pt/C catalyst prepared by the method according to claim 5.

7. A fuel cell electrode comprising the catalyst according to claim 6.

8. A fuel cell comprising the electrode according to claim 7.
